(19) United States
(12) Patent Application Publication
(10) Pub. No.: US 2017/0281273 A1
(43) Pub. Date: Oct. 5, 2017

(54) LASER TREATMENT DEVICE AND METHOD

(71) Applicant: Alma Lasers Ltd., Caesarea (IL)

(72) Inventors: Ziv Karni, Kfar Shmaryahu (IL); Avner Lam, Givat Haim M. (IL); Itay Meir, Haifa (IL); Yevgeny Pens, Kiryat Bialiq (IL)

(21) Appl. No.: 15/473,621

(22) Filed: Mar. 30, 2017

Related U.S. Application Data
(60) Provisional application No. 62/315,034, filed on Mar. 30, 2016.

(51) Int. Cl. A61B 18/20

(52) U.S. Cl. CPC A61B 18/201; A61B 18/20; A61B 2018/00208

(57) ABSTRACT

A laser treatment head in which an elongate applicator is shaped to access a bodily cavity. At the distal end of the applicator is a deflector protected by a window. At a proximal end of the applicator is a drive that can axially displace and rotate the applicator in a helical pattern such that laser beam pulses deflected to the mucosa area applied along a helical path to the wall of the cavity. The drive comprises a stationary hollow shaft inside a rotatable sleeve. The shaft has a helical channel in its outer surface and the sleeve has a longitudinal slot through its wall. A pin attached to the proximal end of the applicator extends through the slot and into the channel. When the sleeve is rotated, the slot advances the pin along the channel, moving the attached applicator in the helical pattern.

### CROSS-REFERENCE TO RELATED APPLICATIONS

The present application takes priority from U.S. Provisional Patent Application 62/315,034, filed Mar. 30, 2016.

### FIELD OF THE INVENTION

The field of the invention is the thermal treatment of tissues lining externally accessible body cavities.

### BACKGROUND OF THE INVENTION

Mucosa is the moist tissue that lines body cavities that are exposed to the external environment. They are at several places continuous with skin: at the nostrils, the mouth, the lips, the eyelids, the ears, the genital area, and the anus. (Herein, the term ‘cavity’ is used generally for these structures, some of which may also be known as ‘canals’.)

There are a number of health problems that are caused by deteriorating laxity, elasticity or tightness of mucous membranes and the underlying adjacent tissues. The following are some of the most common problems: a) involuntary loss of urine called urinary incontinence (UI) among women; b) loss of anal sphincter control; c) vaginal relaxation in women and d) snoring.

Stress urinary incontinence (SUI) is the leakage of urine in response to raised intraabdominal pressure associated with such activities as lifting, sneezing, and coughing. Risk factors for development of this form of incontinence include vaginal delivery, vaginal surgery, inadequate estrogen levels, and advanced age.

Corrective surgery is invasive and expensive and can lead to impairment of normal urinary tract functions. Management options include pelvic floor rehabilitation, behavioral therapy, exercise, pharmacotherapy, intravaginal and intraurethral devices, absorbent products, and external occlusive devices.

Minimally invasive laser treatments are described in U.S. Pat. No. 8,709,057, US20150367142, and WO2014182047. All referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

U.S. Pat. No. 8,709,057 describes a laser device for treatment of mucosa that is moved longitudinally and/or rotationally to guide the laser beam over a target area in multiple passes.

US20150367142 describes a laser device for treatment of a target area within a bodily cavity wherein a laser output element has a laser deflection element that performs a combined axial and rotational movement relative to a guide element in response to motorized rotation of the laser output element relative to the fixed guide element.

A laser-transparent radial distance element (speculum) fixed to the guide element extends into the cavity and serves for the insertion of the laser output element into the vagina and for its free rotational and axial movement therein.

The device described in US20150367142 has a number of drawbacks.

The guide element is a separate component from the laser output element and must be separately held for the duration of the treatment.

The guide element is placed in the external orifice of the cavity, which could add injury, discomfort, or embarrassment for the patient. Furthermore, the guide element may have to be adjusted as the patient moves during treatment, creating more complication for the surgeon and more potential for injury or discomfort for the patient.

The radial distance element, is described as an optional part of the guide element, but appears to actually be required, for reasons that include:
The external threads of the output element would otherwise be exposed inside the cavity, coming into close proximity, or even contact, with the mucosa, creating more potential for injury or infection.
The uncovered mirror could otherwise come into contact with the mucosa or byproducts of the lasing, degrading the mirror's output or harming the mucosa.
The mucosa would otherwise not be held at a known distance from the mirror, degrading control of the laser spot shape and focal area.

Even with a radial distance element, the external threads of the output element remain exposed just outside the cavity, where they can injure or contaminate the tissue.

The radial distance element is made of either laser-transparent material or wire mesh. The structure is not described but can be assumed to present challenges in terms of costs of material and manufacturing.

In the case of laser-transparent material, the entire radial distance element must be composed of the expensive material. In the case of CO2 lasers, it is not known to the inventors a laser-transparent material that can be formed into the required shape.

As for wire mesh, its construction too is not described. It can be assumed to require reinforcing struts as described in earlier U.S. Pat. No. 8,709,057. The struts could obstruct the laser from reaching the mucosa.

Another drawback of the device described in US 20150367142 is that there is no position feedback, therefore accuracy of control is reduced and there can be no detection of motion faults that might require stopping the motion or the lasing to avoid injury to the patient. Nor can the operator set different lasing parameters for different segments of the treatment region.

There is no arrangement for the surgeon to view when manually operating the motor, for example when setting the home and far end points between which the device is to travel during application.

There thus remains a need for improved methods and devices for automated control of laser treatment of externally accessible body cavities.

### SUMMARY OF THE INVENTION

The present invention is directed to a laser treatment head that satisfies this need. The laser treatment head comprises an elongate applicator shaped to access a bodily cavity. At the distal end of the applicator is a deflector protected by a window. At a proximal end of the applicator is a drive that can axially displace and rotate the applicator in a helical pattern so that laser beam pulses deflected to the mucosa are applied along a helical path to the wall of the cavity.

In a preferred embodiment, the drive comprises a stationary hollow shaft inside a rotatable sleeve. The shaft has a helical channel in its exterior and the sleeve has a longitudinal slot through its exterior. A pin attached to the proximal end of the applicator extends through the slot and into the channel. When the sleeve is rotated, the slot causes the pin to ride along the channel, which moves the attached applicator in the helical pattern.

The pin may be fixed in a ring connected to the proximal region of the output element.

There may be a spacer between the drive and the cavity.

The laser may be a CO2 laser.

Preferably, a motor rotates the sleeve, sensors measure at least one of the linear and rotational position of the deflector, and a controller processes the sensor data to operate the laser or the motor.

There may be switches on the laser treatment head that enable the surgeon to manually find and mark one or more linear or rotational endpoints defining the area to be treated.

In a preferred embodiment, the deflector is one of a single flat mirror, a pair of oppositely inclined mirrors, a multiplicity of flat mirrors arranged to form a pyramid and a cone shaped mirror.

In a preferred embodiment, the laser treatment head comprises fractionating elements for fractionating the laser beam into multiple separate beams.

Some of the advantages of the present invention are:
the drive and the applicator are integrated in the laser treatment head.
a spacer, which can be single use, maintains the drive at a safe, hygenic, and constant distance from the patient.
the surgeon can set treatment end points directly from the laser treatment head.
the device can be set to repeatedly pass back and forth between the endpoints, applying treatment along the same path in each pass
linear and rotational position feedback for controlling motion and lasing.
a protective window that moves with the deflector, saving costly laser-transparent material and greatly simplifying manufacture.
the window is part of a sheath, which keeps the mucous at a constant distance from the deflector and can be single use

Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

The invention will now be described with reference to the drawings wherein:

FIG. 1 is a perspective view of an exemplary laser treatment head in accordance with an embodiment of the invention.

FIG. 2 is a view of the laser treatment head with the sheath removed.

FIG. 3 is a view of the laser treatment head with the spacer and casing elements removed.

FIG. 4 is a view of the drive of the laser treatment head.

FIG. 5 is a rotated, detailed partial view of the pin, sleeve, and shaft of the drive of the laser treatment head.

FIG. 6a is a view of the laser treatment head inserted to a distal end point in a patient cavity.

FIG. 6b is a view of the laser treatment head withdrawn to a proximal end point in a patient cavity.

### REFERENCE NUMERALS IN THE DRAWINGS

| | | | |
|---|---|---|---|
| laser treatment | 2 | laser beam | 4 |
| head | | | |
| sheath | 6 | applicator | 8 |
| spacer | 10 | axis | 12 |
| window | 14 | switch | 16 |
| housing | 18 | system connector | 20 |
| deflector | 22 | output element | 24 |
| encoder | 26 | controller | 28 |
| motor | 30 | cog wheel | 32 |
| disk | 34 | socket | 36 |
| drive | 38 | ring | 40 |
| pin | 42 | sleeve | 44 |
| longitudinal slot | 46 | shaft | 48 |
| helical channel | 50 | bodily cavity | 52 |
| proximal region | 54 | distal region | 56 |
| external orifice | 58 | lip | 60 |
| distal end point | 62 | proximal end point | 64 |
| graduation mark | 66 | | |

### DETAILED DESCRIPTION OF THE INVENTION

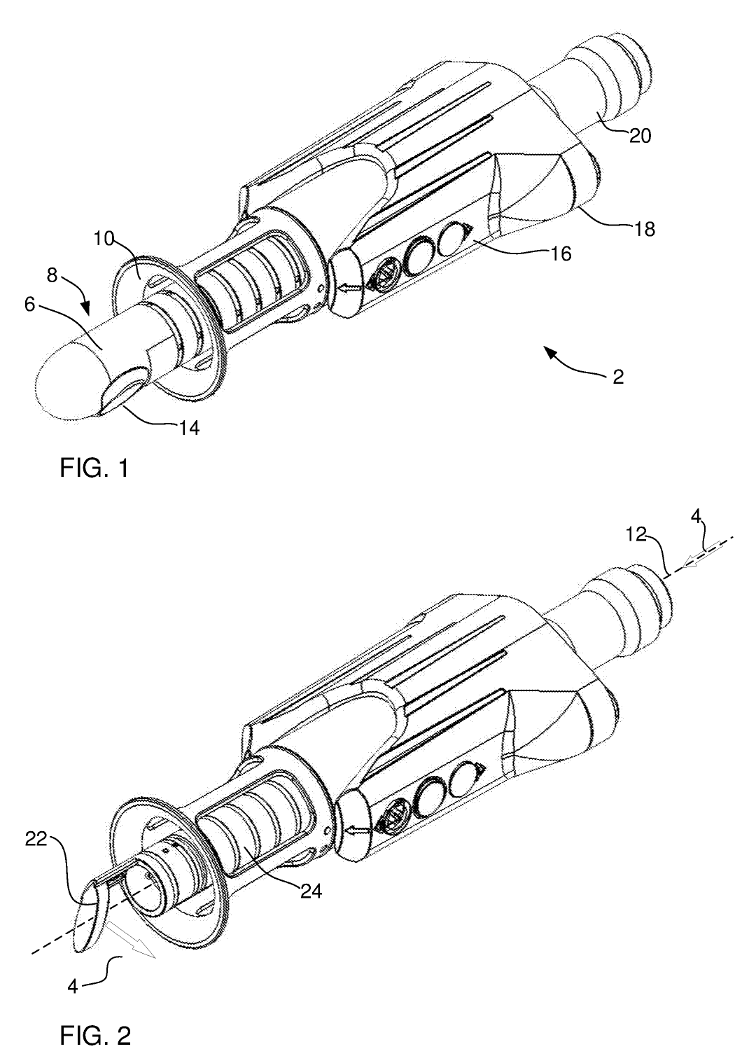

FIG. 1 generally depicts a laser treatment head 2 in accordance with an embodiment of the invention. The laser treatment head is suitable for attachment at system connector 20 to a laser system similar to that described in US20150367142, wherein the laser system is configured to generate a laser beam 4 and guide it to treatment head 2, for example via a segmented arm or optical fiber.

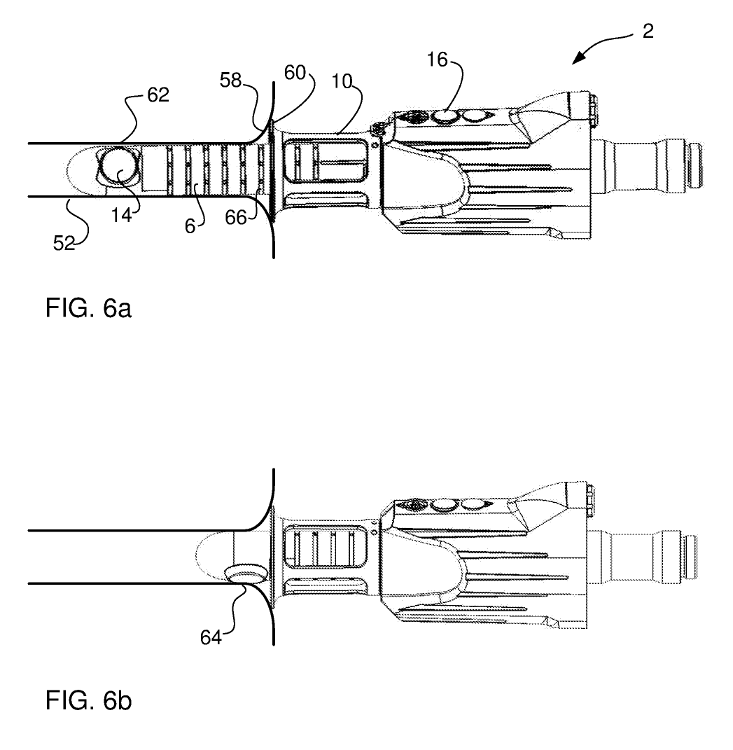

Laser treatment head 2 comprises a drive 38 (FIG. 4) and an applicator 8. Drive 38 is configured to allow passage of laser beam 4 (FIG. 2) to applicator 8 and to impart helical motion on applicator 8. Applicator 8 is configured for insertion into a bodily cavity 52 (FIG. 6), for helical movement therein, and for deflecting the laser beam to the mucosa of the cavity.

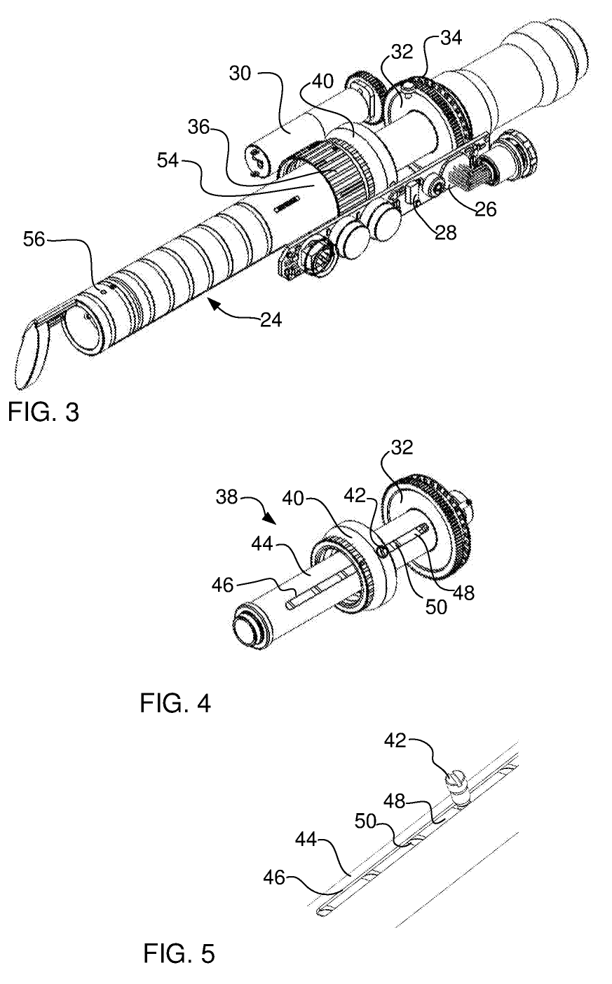

Applicator 8 comprises an output element 24 (FIG. 2, FIG. 3), having a proximal region 54 and a distal region 56, and covered by a sheath 6 (FIG. 1). Output element 24 is attached at its proximal region 54 to drive 38 (FIG. 4) and configured to allow passage of the laser beam 4 to a deflector 22 in distal region 56 that deflects the beam to the adjacent mucosa. Preferably drive 38 and applicator 8 are aligned sequentially along a common longitudinal axis 12 (FIG. 2) and configured so that laser beam 4 can pass parallel to axis 12 from system connector 20 to deflector 22.

Some examples of deflectors are a single flat mirror, a pair of oppositely inclined mirrors, a multiplicity of flat mirrors arranged to form a pyramid and a cone shaped mirror. In a preferred embodiment, the deflector is a single flat mirror that diverts the laser beam at an angle equal to between 30 degrees and 120 degrees from axis 12.

In some embodiments, fractionating elements (not shown) divide laser beam 4 into multiple separate beams. Fractionating elements are well known in the art, including scanners and light directors, for example, those described in US 20140005644, which is incorporated herein by reference.

Sheath 6 may be single use and retained in bayonet socket 36. Sheath 6 comprises material suitable for transvaginal procedures, preferably single-use sterile grade hard plastic for FDA class II devices. Sheath 6 also comprises a window 14 that allows passage of the laser beam 4 from the deflector 22 to the mucosa. Sheath 6 facilitates movement of applicator 8 and keeps deflector 22 at a known and safe distance from the adjacent mucosa. It will be noted that sheath 6 moves integrally with output element 24. As window 14 is in fixed relation to deflector 22, the window need only be as large as the cross sectional area of the laser beam 4 that passes through it (or, in a fractionated embodiment, as the cross sectional area comprising the set of fractionated laser beams).

Window 14 is composed of a material selected for transmission of the wavelength of the laser beam. There is no limitation on the laser beam wavelength, provided appropriate laser-transparent material is used for window 14. In a preferred embodiment, the laser beam 4 is from a CO2 laser (with a wavelength band centering on 10.6 micrometers) and the window 14 comprises Zinc Selenide (ZeSe).

Housing 18 encases drive 38 and comprises a projecting spacer 10, which in operation is maintained with its distal lip 60 in contact with the patient's body adjacent the external orifice 58 of cavity 52 (FIG. 6a). Spacer 10 maintains drive 38 at a constant distance from cavity 52, enabling the surgeon to set treatment end points for movement of deflector 22 and keeping drive 38's moving parts at a safe and hygienic distance from the patient. Spacer 10 also helps keep applicator 8 stable and aligned for movement within cavity 52.

Since spacer 10 is not required for driving applicator 8, lip 60 does not have to contact the patient inside or immediately next to orifice 58. Instead spacer 10 can be formed with a larger diameter so that lip 60 comes into contact with the patient at a remove from orifice 58. Spacer 10 is preferably single use and can be made from the same materials as sheath 6.

With reference to FIGS. 3 to 5, drive 38 comprises stationary hollow shaft 48 inside rotatable sleeve 44. Helical channel 50 extends circumferentially along at least a portion of the exterior of shaft 48. Longitudinal slot 46 extends along at least a portion of the length of sleeve 44. Slot 46 is preferably a through slot, that is, it extends completely through the wall of sleeve 44. Drive 38 further comprises pin 42, which is connected to the proximal region 54 of output element 24 and projects through longitudinal slot 46 and into helical channel 50. In some embodiments pin 42 is an inward projecting element of a ring 40 connected to proximal region 54 of output element 24 and to socket 36.

Motor 30 is configured to impart, through cog wheel 32, rotation to sleeve 44. Disk 34 attached to cog wheel 32 is monitored through encoder 26 by controller 28, which calculates the linear and rotational position of deflector 22. Controller 28 can be programmed to control operation of the motor 30 or the system laser according to the position of deflector 22.

Switches 16 provide the surgeon with manual control of the motor to move deflector 22 to a desired position and to mark the position for controller 28 as an end point. In this manner, she can set linear end points to define the length of a treatment area and rotational end points to define the width of the treatment area. For example, to treat urinary incontinence, end points can be set to restrict the treatment area to a band adjacent the urethra. Switches 16 are located on laser treatment head 2 so that the surgeon can continuous to monitor applicator 8 while setting the end points.

Switches 16 are preferably located adjacent applicator 8, enabling the surgeon when setting end points to track the linear displacement of the applicator by the passage of graduation marks 66 relative to spacer 10.

End points can be set for discrete regions. For example, the controller can be programmed to stop the laser when the deflector 22 reaches a first end point, to resume when the deflector reaches a second end point, to stop again at a third end point, and so forth. This may be useful for treating discrete treatment areas separated by nontreatment areas. Furthermore, the controller can be programmed to change lasing parameters depending on linear or rotary position of the deflector. For example, different segments of the treated cavity can be receive different levels of fluence or pulse widths. This can be useful for adjusting for the type of treatment, the patient's sensitivity level, and anatomical differences, for example, a lower fluence could applied near the external opening of the cavity, where there are relatively more nociceptors.

FIGS. 6a and 6b show exemplary end points 62 and 64. In the example, controller 28 can be programmed to apply the laser automatically along a helical path between a distal end point 62 and a proximal end point 64 with a given number of passes and in one or both directions.

Some embodiments of the invention include a mechanism for rotating the drive 8 itself. In some embodiments, this mechanism includes a second cog wheel behind (first) cog wheel 32, with engagement of motor 30 changeable between the two cog wheels. In other embodiments, this mechanism includes a second motor engaged with a second cog wheel.

Controller 28 can also be programmed to stop motor or laser operation if feedback from encoder 26 indicates a system fault, for example, that deflector 22 has not changed position after a defined number of pulses or that deflector 22 is at an illegal position.

Ring 40 comprises a magnet (not shown) which can be detected by sensors (not shown) located opposite the ends of channel 46, to signal controller 28 that drive 38 has reached the end of its travel and the direction of motor rotation is to be reversed.

In use, the laser treatment head 2 works as follows: controller 28 activates motor 30 to rotate sleeve 44 around stationary shaft 48. Longitudinal slot 46 causes pin 42 and ring 40 to ride along helical channel 50, thereby imparting helical movement to output element 24, including deflector 22. At set intervals, controller 28 fires pulses of laser beam 4, which are deflected by deflector 22 to the target mucosa. The helical pattern of movement of deflector 22 caused by drive 38 cause the laser beam pulses to be applied along a helical path to the wall of the cavity.

Thus, specific embodiments of laser treatment heads have been disclosed. It should be apparent, however, to those skilled in the art that many more modifications besides those already described are possible without departing from the inventive concepts herein. The inventive subject matter, therefore, is not to be restricted except in the spirit of the disclosure. Reference herein to details of the illustrated embodiments is not intended to limit the scope of the claims, which themselves recite those features regarded as essential to the invention.

Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A laser treatment head apparatus for applying a laser beam to mucosa lining a bodily cavity, the apparatus comprising:
(a) an elongate applicator configured for insertion into the cavity, the applicator comprising an output element covered by a sheath;
i. the output element comprising a proximal region and a distal region, the output element configured for the laser beam to pass longitudinally through the proximal region and deflect off a deflector in the distal region towards the mucosa;
ii. the sheath comprising a laser-transparent window arranged so that the laser beam from the deflector passes through it to the mucosa;
and
(b) a drive connected to the applicator, the drive comprising:
i. a fixed hollow shaft with a circumferential helical channel, the shaft configured for the laser beam to pass axially through it to the output element;
ii. a sleeve rotatable about the shaft and comprising a longitudinal slot;
iii. a pin connected to the proximal region of the output element, projecting through the slot and into or through the channel;
whereby, upon rotation of the sleeve, the slot causes the pin to ride along the channel, thereby imparting helical motion to the deflector, and
pulses of the laser beam are applied to the mucosa along a helical path.

2. The apparatus of claim 1 wherein the pin projects inwardly from a ring connected to the proximal region of the output element.

3. The apparatus of claim 1 further comprising a spacer between the drive and the cavity.

4. The apparatus of claim 3 wherein at least one of the sheath and the spacer are single use.

5. The apparatus of claim 1 wherein the laser is a CO2 laser and the window is substantially transparent to light emitted by a CO2 laser.

6. The apparatus of claim 5 wherein the window is comprised of Zinc Selenide (ZeSe).

7. The apparatus of claim 1 further comprising a motor configured to rotate the sleeve.

8. The apparatus of claim 7 further comprising sensors for measuring at least one of the linear and rotational positions of the deflector and a controller programmable to control operation of at least one of a source of the laser beam and the motor in response to sensor data.

9. The apparatus of claim 8 further comprising controls by which a user may manually operate the motor and may mark one or more linear or rotational endpoints defining areas to be treated.

10. The apparatus of claim 9 wherein the controls are arranged to be accessible while monitoring linear displacement of the applicator.

11. The apparatus of claim 9 further comprising a mechanism for rotating the drive, whereby the path may be rotationally offset before or during operation.

12. The apparatus of claim 1 wherein the deflector is one of a single flat mirror, a prism, a pair of oppositely inclined mirrors, a multiplicity of flat mirrors arranged to form a pyramid and a cone shaped mirror.

13. The apparatus of claim 1 further comprising fractionating elements for dividing the laser beam into multiple separate beams.
